(19) United States
(12) Patent Application Publication
(10) Pub. No.: US 2025/0120885 A1
(43) Pub. Date: Apr. 17, 2025

(54) SOLID DELIVERY SYSTEM FOR A SKINCARE ACTIVE INGREDIENT

(71) Applicant: Mana Products, Inc., Long Island City, NY (US)

(72) Inventor: Julio Pina, Woodside, NY (US)

(21) Appl. No.: 18/893,414

(22) Filed: Sep. 23, 2024

Related U.S. Application Data
(60) Provisional application No. 63/539,787, filed on Sep. 21, 2023.

(51) Int. Cl. A61K 8/02; A45D 40/04; A61K 8/368; A61K 8/37; A61K 8/67; A61K 8/92; A61K 8/9728; A61Q 19/00

(52) U.S. Cl. CPC A61K 8/0229; A45D 40/04; A61K 8/025; A61K 8/368; A61K 8/375; A61K 8/676; A61K 8/922; A61K 8/9728; A61Q 19/00; A61K 2800/87

(57) ABSTRACT

Active ingredient delivery systems are provided. Particularly, solid delivery systems for delivering an active skincare agent are provided. Even more particularly, solid serum sticks for delivery an active skincare agent are provided.

### CROSS-REFERENCE TO RELATED APPLICATION

The present disclosure claims priority to Provisional Application No. 63/539,787, entitled SOLID DELIVERY SYSTEM FOR A SKINCARE ACTIVE INGREDIENT, and filed 21 Sep. 2023, the content of which is hereby incorporated by reference herein in its entirety.

### FIELD OF THE INVENTION

The present disclosure relates to novel and advantageous active ingredient delivery systems. Particularly, the present disclosure relates to novel and advantageous solid delivery systems for delivering an active skincare agent. Even more particularly, the present disclosure relates to novel and advantageous solid serum sticks for delivery an active skincare agent.

### BACKGROUND OF THE INVENTION

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Skincare products frequently deliver one or more active ingredients. Such active ingredients may be provided in a water based formulation such as a cream or gel. These water based formulations are not considered an easy to carry and use format. With specific reference to salicylic acid delivery systems, salicylic acid is typically applied using a pimple patch or a paste that is spread using fingers.

Thus, there is a need in the art for an active ingredient delivery system that is solid and convenient for travel.

### BRIEF SUMMARY OF THE INVENTION

The following presents a simplified summary of one or more embodiments of the present disclosure in order to provide a basic understanding of such embodiments. This summary is not an extensive overview of all contemplated embodiments, and is intended to neither identify key or critical elements of all embodiments, nor delineate the scope of any or all embodiments.

The present disclosure, in one or more embodiments, relates to a delivery system for delivering an active skincare ingredient. The delivery system comprises a tube and a composition provided within the tube. The composition may be provided as a solid or semi-solid stick. The tube may be all or partially hollow. The composition may be solid or semi-solid. The composition may comprise a base composition and an active ingredient. The base composition may comprise a wax and a fermented oil. The active ingredient may comprise salicylic acid or vitamin C and may be solubilized. The composition may further comprise a cosmetic component such as a primer. The fermented oil may be one or more of saccharomyces ferment and aspergillus ferment. The wax may be one or more of sunflower wax and tribehenin. The base composition may further comprise light diffusing microspheres. The composition may further comprise a texturizing powder. The texturizing powder may be one or more of lauroyl lysine, silica, and polymethylsilsequioxane.

The delivery systems solubilize an active ingredient in an oil medium and utilize a high concentration of fermented oils, thereby improving aesthetics and efficacy of the formulation. The targeted, travel friendly options make the delivery systems convenient and usable.

While multiple embodiments are disclosed, still other embodiments of the present disclosure will become apparent to those skilled in the art from the following detailed description, which shows and describes illustrative embodiments of the invention. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

### BRIEF DESCRIPTION OF THE DRAWINGS

While the specification concludes with claims particularly pointing out and distinctly claiming the subject matter that is regarded as forming the various embodiments of the present disclosure, it is believed that the invention will be better understood from the following description taken in conjunction with the accompanying Figures, in which:

FIG. 1 illustrates a solid active ingredient delivery system, in accordance with one embodiment.

FIG. 2 illustrates a solid active ingredient delivery system, in accordance with a further embodiment.

FIG. 3 is an ingredient list for a solid delivery system for a skincare active ingredient in accordance with a first embodiment.

FIG. 4 is an ingredient list for a solid delivery system for a skincare active ingredient in accordance with a second embodiment.

### DETAILED DESCRIPTION

The present disclosure relates to novel and advantageous active ingredient delivery systems. Particularly, the present disclosure relates to novel and advantageous solid delivery systems for delivering an active skincare agent. Even more particularly, the present disclosure relates to novel and advantageous solid serum sticks for delivery an active skincare agent.

The delivery systems, also referred to as treatment systems, treatment sticks, or serum sticks, disclosed herein may be configured for delivery of one or more active ingredients. Three embodiments are described—a delivery system for delivery of vitamin C, a delivery system for delivery of a salicylic acid, and a delivery system for delivery of a salicylic acid with a primer. It is to be appreciated that the solid delivery systems may be used for delivery of other active ingredients. In some embodiments, the delivery system may delivery one or more active ingredients and also have cosmetic properties, such as imparting a tint to skin on which it is applied.

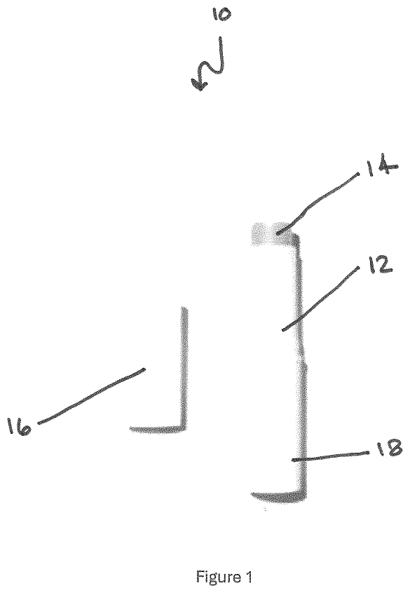

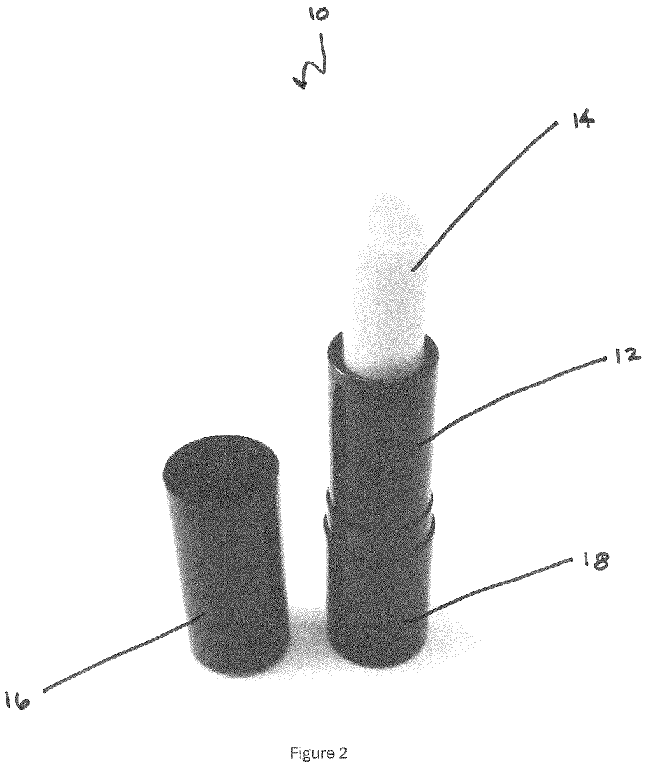

FIGS. 1 and 2 illustrate a sample delivery systems 10. As shown, the delivery system comprises a tube 12 having a composition provided therein. The composition may be formulated as a solid or semi-solid stick 14 such that it can be moved in and out of the tube without losing its structure. The stick may have length and diameter chosen for suitability for a specific application. A cap 16 may be provided for enclosing the stick 14 within the tube 12.

FIG. 1 illustrates an embodiment having a relatively large diameter the diameter to facilitate easy broad coverage application. may be relatively large. For example, the stick may have a diameter similar to a foundation stick, ranging from about 15 mm to about 25 mm.

FIG. 2 illustrates an embodiment having a relatively small diameter for targeted application of an active ingredient. For example, the stick may have a diameter similar to a lipstick, ranging from about 9 mm to about 16 mm.

The tube may be hollow throughout or hollow through a portion thereof. In some embodiments, approximately 3.5 grams/0.12 oz. to 17 grams/0.6 oz. of composition may be provided within the tube. In alternative embodiments, more or less composition may be provided within the tube. The tube may be provided with a mechanism for extending the stick from the tube and, optionally, retracting the stick back into the tube. For example, the stick may rest on a platform that may be raised or lowered using an elevator actuated by manipulating part of the tube. For example, a lower portion 18 of the tube 12 may be rotated to actuate the elevator.

In general, the composition may comprise a base composition and an active ingredient. The base composition may be similar between different embodiments. Variation of the base composition may be modified based on the active ingredients being delivered or desired uses of the stick. For example, the composition may be provided with a pigment such that it may be used as a foundation stick, a blush stick, a lipstick, etc.

The composition may comprise a hydro stick formulation. A challenge with skincare is that skin is hydrophobic on the outside, meaning that water will not penetrate the skin. In order to enhance efficacy of absorption of the active ingredient by the skin, formulations provided herein may include an oil.

Silicones are synthetic compounds which contain siloxane bonds. Silicone compounds have become common ingredients in skin care. Silicones can act as a barrier and trap oil, dirt and dead skin cells. Further, silicones may block subsequently applied ingredients from reaching the skin, rendering anything applied after a silicone product ineffective.

The compositions described herein use high fermentation ingredients to provide performance attributes and dry down of the formulation. In various embodiments, the high fermentation ingredients comprise approximately 25% of the formulation. The high fermentation ingredients allow for a lower usage of silicone than traditional in skincare products. Fermented oils can boost ingredient penetration into the skin, can prolong moisturizing effects, and can increase antioxidant activity, among other beneficial effects.

Ingredients used in the delivery system composition may include a wax and a fermented oil. In some embodiments, the composition comprises sunflower oil, hemisqualane, saccaroymycea ferment and aspergillus ferment, and light diffusing microspheres.
Sunflower Oil is a non-comedogenic oil that is suitable for all skin types. It contains linoleic acid to help maintain, replenish, and strengthen skin's natural barrier.
Hemisqualane ˜20% is light-textured and highly emollient. It is quickly absorbed and helps rejuvenate parched skin without leaving it greasy.
Saccharomyces has moisturizing and antioxidant properties. Aspergillus ferment has skin-brightening benefits, has antioxidant and skin barrier strengthening properties, and can enhance the bioactivity of other skincare ingredients. Saccharomyces Ferment and Aspergillus Ferment can be combined into a synergistic blend of natural fermented emollients to help to create a light, dry, fast-absorbing formula.
Light Diffusing Microspheres, including natural and synthetic spherical powders, help speed absorption and enhance feel on skin.

Three embodiments are specifically described herein. It is to be appreciated that these are exemplary only and different active ingredients may be delivered using a delivery system as disclosed herein. The delivery systems solubilize an active ingredient in an oil medium and utilize a high concentration of fermented oils, thereby improving aesthetics and efficacy of the formulation. The targeted, travel friendly options make the delivery systems convenient and usable.

### Solid Delivery System for Delivery of Salicylic Acid

A first embodiment comprises a treatment system for delivery of salicylic acid. The salicylic acid delivery system enhances absorption of salicylic acid by the skin by using a high percentage of fermented oils. The salicylic acid operates to clean out pores of the skin. The salicylic acid delivery system may contain salicylic acid at approximately 2%. An ester, such as octadecanoyl, may be used to solubilize the salicylic acid without precipitating it out. The salicylic acid gently exfoliates, helping to decongest clogged ores, blackheads, and pimples.

In some embodiments, BV-OSC 1% Vitamin C may be combined with the salicylic acid to additionally help reduce dark spots, brighten, and even tone.

The salicylic acid is solubilized in an oil medium and utilizing a high concentration of fermented oils in order to improve the aesthetics and the efficacy of the formula. The fermented oils may be saccharomyces ferment and aspergillus ferment, a mixture at 5-8% and c13-15 alkane fermented from sugarcane.

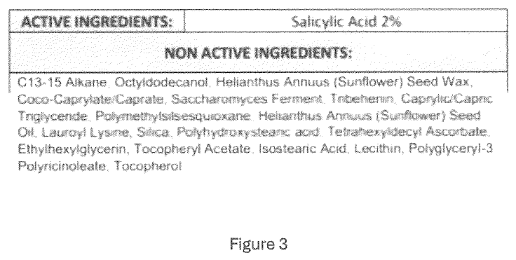

FIG. 3 illustrates an ingredient list for a solid delivery system for delivery of salicylic acid, in accordance with one embodiment.

### Method for Making Salicylic Acid Composition:

Phases A-E are utilized in making a salicylic acid delivery system. Phase A comprises an organo-clay premade mixture that is utilized as a suspending and stabilizing agent. Phase B comprises one or more waxes such as sunflower wax and tribehenin (both are naturally derived). Phase C comprises texturizing powders (lauroyl lysine, silica, and polymethylsilsesquioxan). Phase D comprises a salicylic acid solution. Phase E comprises complementary actives such as ceramides and fermented oil and is beneficial for aesthetics improvement. Phase E may also contain a preservative booster.

A method for making a salicylic acid composition for the delivery system is outlined below. More or fewer steps may be used as would be appreciated by one skilled in the art.
Prepare Phase A in a primary beaker and begin heating the formula between 80-85° C., mix until uniform.
Add Phase B to Phase A and continue to heat between 80-85° C. and mix until the waxes of melting points ranging from 55-65° C. and 75-8°5 C are completely dissolved. Mix until homogeneous.
Add Phase C to the formula and assure all texturizing powders are fully dispersed.
Add Phase D to mixture and mix until uniform.
Add Phase E to mixture and mix until uniform.

### Solid Delivery System for Delivery of Salicylic Acid and a Color Cosmetic

A second embodiment comprises a hybrid skincare and color cosmetic delivery system. More specifically, this embodiment comprises solid delivery system for delivery of salicylic acid while applying a color cosmetic, such as a primer, to the skin. The hybrid skincare and color cosmetic delivery system includes a powder for priming benefits.

In one formulation, the solid system for delivery of salicylic acid and a color cosmetic comprises salicylic acid 0.50%, U-Oliosome Cera 1, and 25% fermented oil blend. The salicylic acid gently exfoliates, helping to decongest clogged pores, blackheads, and pimples. The U-Oliosome Cera 1, encapsulated ceramide NP in a reverse micelle, helps penetration into skin lipid layer to help boost skin barrier function and protection. The fermented oil blend is light-textured and highly emollient, and is quickly absorbed by the skin to help rejuvenate parched skin without leaving it greasy.

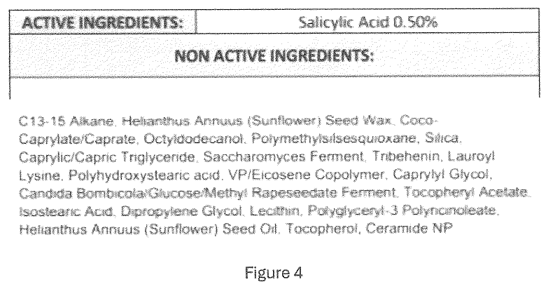

FIG. 4 illustrates an ingredient list for a hybrid skincare and color cosmetic delivery system including salicylic acid.

### Method for Making Hybrid Skincare and Color Cosmetic Composition:

Phases A-E are utilized in making a salicylic acid and color cosmetic delivery system. Phase A comprises an organo-clay premade mixture that is utilized as a suspending and stabilizing agent. Phase B comprises one or more waxes such as sunflower wax and tribehenin (both are naturally derived). Phase C comprises texturizing powders (lauroyl lysine, silica, and polymethylsilsesquioxan). Phase D comprises a salicylic acid solution. Phase E comprises complementary actives such as ceramides and fermented oil and is beneficial for aesthetics improvement. Phase E may also contain a preservative booster.

A method for making a salicylic acid and color cosmetic composition for the delivery system is outlined below. More or fewer steps may be used as would be appreciated by one skilled in the art. Pigment may be added as appropriate.
In a side beaker, prepare the active phase in a separate beaker by heating octyldodecanol and salicylic acid between 50-60° C. to form Phase D. Assure the salicylic acid is completely solubilized. Once clear and uniform, drop the temperature until the solution reaches room temperate.
Prepare Phase A in a primary beaker and begin heating the formula between 80-85° C., mix until uniform.
Add Phase B to phase A and continue to heat between 80-85° C. and mix until the waxes of melting points ranging from 55-65° C. and 75-85° C. are completely dissolved. Mix until homogeneous.
Add Phase C to the formula and assure all texturizing powders are fully dispersed.
Add Phase D to ABC and mix until uniform.
Add Phase E to ABC and mix until uniform.

### Solid Delivery System for Delivery of Vitamin C

A third embodiment comprises a solid delivery system for delivery of vitamin C. The vitamin C delivery system enhances absorption of vitamin C by the skin by using a high percentage of fermented oils. The vitamin C treats hyperpigmentation.

In one formulation, a solid delivery system for delivery of vitamin C comprises BV-OSC (vitamin c oil soluble) 5%. This includes a stable, bio-available, oil-soluble form of vitamin C to help reduce dark spots, brighten, and even tone.

In one embodiment, the vitamin C delivery system contains vitamin C at approximately 5%.

### Method of Process for Making a Vitamin C Composition:

Phases A-C and E are utilized in making a vitamin C composition. Phase A comprises an organo-clay premade mixture that is utilized as a suspending and stabilizing agent. Phase B comprises one or more waxes such as sunflower wax and tribehenin (both are naturally derived). Phase C comprises texturizing powders (lauroyl lysine, silica, and polymethylsilsesquioxan). Phase E comprises complementary actives such as ceramides and fermented oil and is beneficial for aesthetics improvement. Phase E may also contain a preservative booster.

A method for making a salicylic acid and color cosmetic composition for the delivery system is outlined below. More or fewer steps may be used as would be appreciated by one skilled in the art.
Prepare Phase A in a primary beaker and begin heating the formula between 80-85° C., mix until uniform.
Add Phase B to phase A and continue to heat between 80-85° C. and mix until the waxes of melting points ranging from 55-65° C. and 75-85° C. are completely dissolved. Mix until homogeneous.
Add Phase C to the formula and assure all texturizing powders are fully dispersed.
Add Phase E to ABC mixture and mix until uniform.

As used herein, the terms “substantially” or “generally” refer to the complete or nearly complete extent or degree of an action, characteristic, property, state, structure, item, or result. For example, an object that is “substantially” or “generally” enclosed would mean that the object is either completely enclosed or nearly completely enclosed. The exact allowable degree of deviation from absolute completeness may in some cases depend on the specific context. However, generally speaking, the nearness of completion will be so as to have generally the same overall result as if absolute and total completion were obtained. The use of “substantially” or “generally” is equally applicable when used in a negative connotation to refer to the complete or near complete lack of an action, characteristic, property, state, structure, item, or result. For example, an element, combination, embodiment, or composition that is “substantially free of” or “generally free of” an element may still actually contain such element as long as there is generally no significant effect thereof.

To aid the Patent Office and any readers of any patent issued on this application in interpreting the claims appended hereto, applicants wish to note that they do not intend any of the appended claims or claim elements to invoke 35 U.S.C. § 112(f) unless the words “means for” or “step for” are explicitly used in the particular claim.

Additionally, as used herein, the phrase “at least one of [X] and [Y],” where X and Y are different components that may be included in an embodiment of the present disclosure, means that the embodiment could include component X without component Y, the embodiment could include the component Y without component X, or the embodiment could include both components X and Y. Similarly, when used with respect to three or more components, such as “at least one of [X], [Y], and [Z],” the phrase means that the embodiment could include any one of the three or more components, any combination or sub-combination of any of the components, or all of the components.

In the foregoing description various embodiments of the present disclosure have been presented for the purpose of illustration and description. They are not intended to be exhaustive or to limit the invention to the precise form disclosed. Obvious modifications or variations are possible in light of the above teachings. The various embodiments were chosen and described to provide the best illustration of the principals of the disclosure and their practical application, and to enable one of ordinary skill in the art to utilize the various embodiments with various modifications as are suited to the particular use contemplated. All such modifications and variations are within the scope of the present disclosure as determined by the appended claims when interpreted in accordance with the breadth they are fairly, legally, and equitably entitled.

What is claimed is:

1. A delivery system for delivering an active skincare ingredient, the delivery system comprising:
a tube; and
a composition provided within the tube, wherein the composition comprises:
a base composition comprises a wax and a fermented oil; and
an active ingredient wherein the active ingredient is solubilized.

2. The delivery system of claim 1, wherein the active ingredient is salicylic acid.

3. The delivery system of claim 2, wherein the composition further comprises a cosmetic component.

4. The delivery system of claim 3, wherein the cosmetic component is a primer.

5. The delivery system of claim 1, wherein the active ingredient is vitamin C.

6. The delivery system of claim 1, wherein the fermented oil comprises one or more of saccharomyces ferment and aspergillus ferment.

7. The delivery system of claim 1, wherein the wax comprises one or more of sunflower wax and tribehenin.

8. The delivery system of claim 1, further comprising light diffusing microspheres.

9. The delivery system of claim 1, wherein further comprising a texturizing powder.

10. The delivery system f claim 9, wherein the texturizing powder comprises one or more of lauroyl lysine, silica, and polymethylsilsequioxane.

11. The delivery system of claim 1, wherein the tube is hollow.

12. The delivery system of claim 1. wherein the composition is solid.

13. The delivery system of claim 1. wherein the composition is semi-solid.
